(19) United States
(12) Patent Application Publication
(10) Pub. No.: US 2013/0217892 A1
(43) Pub. Date: Aug. 22, 2013

(54) Use Of N-Substituted 2-Pyrrolidone-4-Carboxylic Acid Esters And Solvents

(71) Applicants: Andreas Wacker, Mannheim (DE); Joachim Hess, Hofheim (DE); Rainer Kupfer, Hattersheim (DE); Alexander Roesch, Oppenheim (DE)

(72) Inventors: Andreas Wacker, Mannheim (DE); Joachim Hess, Hofheim (DE); Rainer Kupfer, Hattersheim (DE); Alexander Roesch, Oppenheim (DE)

(73) Assignee: CLARIANT FINANCE (BVI) LIMITED, Tortola (VG)

(21) Appl. No.: 13/822,960

(22) PCT Filed: Sep. 14, 2011
(86) PCT No.: PCT/EP11/04613

§ 371 Date: Mar. 27, 2013

(30) Foreign Application Priority Data
Sep. 16, 2010 (DE) 10 2010 046 680.8

(51) Int. Cl. C07D 207/277; A01N 25/02

(52) U.S. Cl. CPC C07D 207/277; A01N 25/02
USPC 548/531

(57) ABSTRACT

The invention relates to the use of one or more N-substituted 2-pyrrolidone-4-carbonic acid esters of formula (1), where R1 and R2 independently represent linear, branched, or cyclic C1-C6 alkyl, as (a) solvent(s). Said compounds can be used as solvents in industrial applications, e.g. for the production of varnishes and paints, in chemical syntheses, for cleaning or degreasing, in petrochemical processes, in the electronics and photovoltaic industries, or in pesticide preparations, for example.

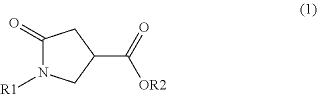

Use of N-substituted 2-pyrrolidone-4-carboxylic acid esters as solvents

The invention relates to the use of certain N-substituted pyrrolidone carboxylic acid esters as solvents.

Being a polar, aprotic and broadly applicable organic solvent with a low viscosity which is homogeneously miscible with water and other organic solvents, N-methylpyrrolidone (NMP) has become established in research and in technology for many applications. On account of these manifold use options, the annual consumption of NMP is several tens of thousands of tonnes worldwide.

However, the toxicological properties of NMP are disadvantageous. Besides the irritancy effect and the associated labeling with the R phrases R36 to 38, this is primarily the embryotoxic effect which, according to the 31^{st} adaption guideline (31^{st} ATP, Adaption to technical progress of council directive 67/548/EEC), leads to the labeling of NMP with the hazard symbol “T” for “toxic”. The same applies to preparations which comprise NMP in amounts of >5% by weight. Moreover, NMP is a classic petrochemical product which is produced on an industrial scale exclusively from petroleum-based raw materials.

There is therefore a need for alternative solvents, ideally based on renewable raw materials, which have comparable or better properties than NMP and are less toxicologically objectionable. The suitability of a possible NMP replacement is determined essentially by its physicochemical properties. In the case of a solvent for industrial applications, such as in particular the chemical industry, the paints and coatings industry, electronics industry or agrochemical industry, these are e.g. setting point and boiling point, flash point, viscosity, polarity, inertness, dissolving capacity, miscibility with water and other solvents.

It was therefore an object of the present invention to provide novel toxicologically less objectionable and environmentally friendly polar solvents as replacement for NMP.

Surprisingly, it has now been found that this object is achieved by certain N-substituted pyrrolidonecarboxylic acid esters.

The invention therefore provides the use of one or more N-substituted 2-pyrrolidone-4-carboxylic acid esters of the formula (1)

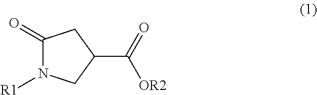

in which
R1 and R2 independently of one another are a linear, branched or cyclic C₁-C₆-alkyl, as solvents.

Preferably, R1 and R2 in the compounds of the formula (1) independently of one another are linear or branched C₁-C₆-alkyl.

Particularly preferably, R1 and R2 in the compounds of the formula (1) independently of one another are methyl, ethyl, n-propyl, isopropyl, n-butyl or isobutyl.

Particularly preferably, in the compounds of the formula (1),
R1 and R2 are methyl or
R1 is methyl and R2 is isobutyl or
R1 is n-butyl and R2 is methyl or
R1 is isobutyl and R2 is methyl.

The preparation of the compounds of the formula (1) is possible for example by reaction of itaconic acid with alkylamines and subsequent esterification or by direct reaction of itaconic acid esters with alkylamines and is described in the literature (Wu; Feldkamp; Journal of Organic Chemistry; vol. 26; (1961); p. 1519 or Arvanitis, Motevalli, Wyatt, Tetrahedron Lett. 1996, 37, 4277-4280).

DE 24 52 536 describes N-alkylpyrrolidone derivatives synthesized over a plurality of stages and their use as pharmaceutical active ingredients. Various short-chain N-alkyl-2-pyrrolidone-4-carboxylic acid esters are formed here as intermediates in the synthesis of these active ingredients.

EP 1 342 759 discloses an ink-jet ink which, besides water, comprises a water-soluble ester or amide of pyrrolidone-2-carboxylic acid substituted at different points of the heterocyclic ring, the pyrrolidone carboxylic acid derivatives serving to improve the print quality.

EP 2 028 247 describes the use of N-alkyl-2-pyrrolidone-4-carboxylic acid esters as gas hydrate inhibitors.

WO 2010/033447 discloses the use of various heterocycles, including N-alkyl-2-pyrrolidone-4-carboxylic acid esters as EP/AW additive in lubricants.

EP 2 193 782 and EP 2 193 784 disclose cosmetic preparations which comprise a UV absorber and an N-alkylpyrrolidonecarboxylic acid ester.

DE 10 2009 043 122.5 describes long-chain (R1>C7) representatives of N-alkyl-2-pyrrolidone-4-carboxylic acid, including their esters and their use in crop protection formulations, for example as adjuvant or emulsifier. The use of short-chain derivatives is not described therein.

The use of compounds of the formula (1) as solvents has not been described to date.

In contrast to long-chain alkylpyrrolidonecarboxylic acid esters (see formula (1), but where R1>C7), which are obtained by reaction of relatively long-chain alkylamines or fatty amines (R1>C7), N-alkyl-2-pyrrolidone-4-carboxylic acid esters prepared on the basis of short-chain amines (R1<C7) are characterized by a high polarity and solubility in water. This applies in particular if the alkyl group of the ester function is likewise short-chain (R2<C7). These properties are essential for solvents which are intended to serve as NMP replacement.

Moreover, the compounds of the formula (1) have further advantageous physicochemical properties. By virtue of their low setting point of considerably lower than 0° C., they can also be used as solvents at low temperatures without solidifying. This is advantageous for example in the winter or cold regions both during use and also during storage. The high boiling point brings about a low vapor pressure and a high flashpoint (typically >100° C.), meaning that safety-related advantages also support the use of these solvents. A further important requirement placed on environmentally friendly solvents nowadays is a low content of volatile organic compounds (Volatile Organic Solvents, VOC), which is met by the compounds of the formula (1). An established method of determining the VOC content is a gas chromatographic analysis in accordance with DIN EN ISO 11890-2. Preferably, the VOC content of the compounds of the formula (1) in accordance with DIN EN ISO 11890-2 is less than 5% by weight, particularly preferably less than 1% by weight and especially preferably less than 0.5% by weight.

The compounds of the formula (1) are expediently prepared from itaconic acid or derivatives thereof. Itaconic acid is obtained on an industrial scale from sugar and, according to the study of the same name ordered by the US Department of Energy from 2004, counts among the “Top Value Added Chemical From Biomass” (http://www.nrel.gov/docs/fy04osti/35523.pdf). The compounds of the formula (1) derived therefrom can therefore be prepared in an environmentally friendly manner on the basis of renewable raw materials.

Preferably, the use according to the invention of the compounds of the formula (1) is as solvents in industrial applications.

Particularly preferably, the use is in the coatings, paint, printing ink, agrochemical, pharmaceutical, petrochemical, electronics or photovoltaic industries. Particular preference is given to the uses as solvents for binder systems, in cleaners, in paint strippers or graffiti removers, in gas scrubbing, for adhesives or adhesive removers, for degreasing, for extraction or purification of substance mixtures, in chemical or pharmaceutical synthesis, and for the manufacture of pigments or in pigment preparations.

In a particularly preferred embodiment of the invention, the compounds of the formula (1) are used as solvents in the manufacture of coatings and paints. On account of their very good solvent properties and the high chemical resistance, the highly diverse components of a coating or of a paint can either dissolve or disperse in the compounds of the formula (1). The high boiling point additionally has a positive influence on the progress and curing of the coatings or paints.

Particular preference is given to the use of the compounds of the formula (1) as solvents in the production of binder dispersions, as are used in the coatings industry, in particular of polyurethane dispersions (PUDs). PUDs are free-flowing two-phase systems consisting of water and a polymer, i.e. a dispersed plastic which belongs to the class of polyurethanes, and also optionally further components. Compounds of the formula (1) function as cosolvent in PUDs, usually in the order of magnitude of 5 to 15% by weight. Furthermore, particular preference is given to the use of the compounds of the formula (1) as solvents in the production of thermally curable coatings for electrical insulation, in particular of wire enamels.

Wire enamels are solutions of polymers in solvent mixtures and are usually baked at 300 to 600° C., during which the solvent evaporates, the polymers crosslink and insoluble enamel films are formed. Wire enamels based on polyurethanes are formed by the addition of hydroxyl compounds, preferably hydroxy-group-containing polyesters, onto oligomeric aromatic isocyanates, from e.g. trimethylolpropane and toluyl isocyanate. A premature reaction of the single-component enamels during transportation and storage is prevented by the blocking of the isocyanate with phenol. At temperatures above 180° C., phenol is cleaved off, and the isocyanate formed crosslinks with the hydroxyl component to give the polyurethane. For winding wires in coils, transformers, relays and engines, coatings with polyester imides as film formers are often used. Depending on the composition and selection of the raw materials, films with different thermal, mechanical and dielectric properties are obtained. Polyester imides are prepared from ethylene glycol, tris-2-hydroxyethylisocyanurate (THEIC), dimethyl terephthalate, trimellitic anhydride, diaminodiphenylmethane in a polycondensation reaction. Commercially available polyester imides have a hydroxide mass ratio between 100 and 300 mg/g and a molar mass below 5000 g/mol and are provided as ca. 40% strength by weight solutions in a solvent.

In a further particularly preferred embodiment of the invention, the compounds of the formula (1) are used as solvents in chemical synthesis. On account of the high solubilities of many reactants, many reactions can be carried out in solution, i.e. homogeneously, which increases reaction rate, yield and selectivity. As a result of the high boiling point, reactions can take place at a high temperature, which accelerates the conversions. The chemical inertness of the compounds of the formula (1) permits the manifold use in a variety of syntheses. Examples of reactions which can be carried out in the compounds of the formula (1) are substitution reactions, condensations or addition reactions. Preferred fields of use are the synthesis of pharmaceutical or agrochemical active ingredients, and also preproducts thereof, and the synthesis of organic pigments. During pigment production, the actual synthesis steps can be carried out in the compounds of the formula (1), such as condensation reactions, for example in the case of the production of heterocyclic pigments or of azo condensation pigments. Moreover, the after-treatment (the “finish”) of pigments can take place in the compounds of the formula (1). Here, the pigment suspension produced after the pigment synthesis or the crude pigment is admixed with the solvent and subjected to a temperature treatment, during which water or other solvents are optionally distilled off. During this process, physical properties such as the crystal modification and particle size distribution of the organic pigments are influenced in a targeted manner in order to optimize the application-related properties as colorants.

In a further particularly preferred embodiment of the invention, the compounds of the formula (1) are used as solvents for cleaning or degreasing. On account of the high dissolving capacity, a very wide variety of undesired substances, such as fats, oils, soot, adhesive residues or resins can be removed from surfaces, for example prior to coating by painting or vapor deposition. Likewise possible is the use in order to remove existing coatings from surfaces, such as during paint stripping or graffiti removal. The high boiling point of the solvents reduces the vaporization and leads to a better efficiency over extended exposure times and also to a lesser impact on the environment due to solvent emissions.

The high flashpoint of the solvents increases the industrial safety and leads to an avoidance of explosion or fire risks. As well as using the pure compounds of the formula (1) as solvents, they can also expediently be used together with other constituents in the form of a cleaner formulation. Other components of this formulation are usually water or other solvents, surfactants, dyes or thickeners.

In a further particularly preferred embodiment of the invention, the compounds of the formula (1) are used as solvents in petrochemical processes, particularly for the separation and purification of hydrocarbons. During the refining of petroleum and the subsequent further processing in the cracking process, at times complex mixtures of different hydrocarbons are produced at various points which have to be purified by methods such as distillation or extraction. Polar solvents such as NMP can be used in these processes as extractants. Often, mixtures are used here which comprise water or other solvents in order to achieve the highest possible selectivity for the particular process step. The compounds of the formula (1) can likewise be used as formulation auxiliaries of additives which are used for the recovery and the transportation of petrochemical raw materials such as oil and gas. Mention may be expressly made of additives for preventing corrosion, gas hydrate formation, boiler scale formation (“scale inhibitors”) and for the deposition of asphaltenes.

In a further particularly preferred embodiment of the invention, the compounds of the formula (1) are used as solvents in the electronics and photovoltaic industry. In the manufacture of wafers, integrated circuits (ICs) such as microprocessors or storage chips and printed circuit boards, solvents of very high purity are used in various production steps. In particular, a very low content of metal traces plays a decisive role here. Compounds of the formula (1) can be provided in a grade sufficient for this application by means of corresponding purification steps such as distillation. In particular, compounds of the formula (1) are suitable as solvents in the following process steps: the purification or degreasing of silicon wafers for producing ICs prior to the start of the photolithographic process and in the various steps of the subsequent photolithography. The compounds of the formula (1) are thus particularly preferably suitable as solvents in photolithography. The solvent here can assume various functions, as a constituent of the photoresist itself, in the developer liquid for removing the unexposed negative photoresist or the exposed positive photoresist, in photoresist strippers, and also in cleaning liquids in order to remove excess etching fluid.

In a further particularly preferred embodiment of the invention, the compounds of the formula (1) are used as solvents in pesticide preparations.

The pesticide preparations preferably comprise
a) one or more pesticides and
b) one or more N-substituted 2-pyrrolidone-4-carboxylic acid esters of the formula (1).

Within the context of the present invention, “pesticides” are understood as meaning herbicides, fungicides, insecticides, acaricides, bactericides, molluscicides, nematicides and rodenticides, and also phytohormones. Preference is given to herbicides, insecticides and fungicides.

Preferred fungicides are aliphatic nitrogen fungicides, amide fungicides such as acylaminoacid fungicides or anilide fungicides or benzamide fungicides or strobilurin fungicides, aromatic fungicides, benzimidazole fungicides, benzothiazole fungicides, carbamate fungicides, conazole fungicides such as imidazoles or triazoles, dicarboximide fungicides, dithiocarbamate fungicides, imidazole fungicides, morpholine fungicides, oxazole fungicides, pyrazole fungicides, pyridine fungicides, pyrimidine fungicides, pyrrole fungicides, quinone fungicides.

Preferred herbicides are amide herbicides, anilide herbicides, aromatic acid herbicides such as benzoic acid herbicides or picolinic acid herbicides, benzoylcyclohexanedione herbicides, benzofuranylalkylsulfonate herbicides, benzothiazole herbicides, carbamate herbicides, carbanilate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, imidazolinone herbicides, nitrile herbicides, organophosphorus herbicides, oxadiazolone herbicides, oxazole herbicides, phenoxy herbicides such as phenoxyacetic acid herbicides or phenoxybutanoic acid herbicides or phenoxypropionic acid herbicides or aryloxyphenoxypropionic acid herbicides, pyrazole herbicides such as benzoylpyrazole herbicides or phenylpyrazole herbicides, pyridazinone herbicides, pyridine herbicides, thiocarbamate herbicides, triazine herbicides, triazinone herbicides, triazole herbicides, triazolone herbicides, triazolopyrimidine herbicides, uracil herbicides, urea herbicides such as phenylurea herbicides or sulfonylurea herbicides.

Preferred insecticides are carbamate insecticides, such as benzofuranyl methylcarbamate insecticides or dimethylcarbamate insecticides or oximecarbamate insecticides or phenyl methylcarbamate insecticides, diamide insecticides, insect growth regulators, macrocyclic lactone insecticides such as avermectin insecticides or milbemycin insecticides or spinosyn insecticides, nereistoxin analog insecticides, nicotinoid insecticides such as nitroguanidine nicotinoid insecticides or pyridylmethylamine nicotinoid insecticides, organophosphorus insecticides such as organophosphate insecticides or organothiophosphate insecticides or phosphonates insecticides or phosphoramidothioate insecticides, oxadiazine insecticides, pyrazole insecticides, pyrethroid insecticides such as pyrethroid ester insecticides or pyrethroid ether insecticides or pyrethroid oxime insecticides, tetramic acid insecticides, tetrahydrofurandione insecticides, thiazole insecticides.

Particularly preferably, the one or more pesticides of component a) of the pesticide preparations is or are selected from the group consisting of aryloxyphenoxypropionic acid herbicides, benzoylcyclohexanedione herbicides, triazolopyrimidine herbicides, strobilurin fungicides, triazole fungicides, nicotinoid insecticides and pyrethroid insecticides.

Particularly preferably, the one or more pesticides of component a) of the pesticide preparations is or are selected from the group consisting of trifloxystrobin, tebuconazole, pendimethalin, triadimefon and trifluralin.

The preparation of the pesticide preparations is possible in various ways depending on the type of formulation and is sufficiently known to the person skilled in the art.

The pesticide preparations comprise the one or more pesticides of component a) in amounts of from preferably 0.1 to 75% by weight, particularly preferably form 5 to 50% by weight and particularly preferably from 10 to 40% by weight. This quantitative data is based on the total weight of the pesticide preparations.

Furthermore, the pesticide preparations comprise the one or more compounds of the formula (1) preferably in amounts of from 0.1 to 99% by weight, particularly preferably from 5 to 75% by weight and especially preferably from 10 to 50% by weight. This quantitative data is based on the total weight of the pesticide preparations.

The pesticide preparations can comprise one or more auxiliaries which perform a wide variety of functions. Examples of auxiliaries according to their function are thickeners, additional solvents, dispersants, emulsifiers, preservatives, adjuvants, binders, thinners, disintegrants, wetting agents, penetration promoters, low-temperature stabilizers, colorants, antifoams, antioxidants, crystallization inhibitors, antifreezes or humectants.

Moreover, the pesticide preparations can comprise one or more agrochemical salts, preferably potassium or ammonium salts.

Preferably, the pesticide preparations are free from N-methylpyrrolidone.

### EXAMPLES

The invention is illustrated below by reference to examples, although these should in no way be regarded as a limitation.

### Example 1

### Preparation of Methyl N-methyl-2-Pyrrolidone-4-Carboxylate

239.9 g of N-methyl-2-pyrrolidone-4-carboxylic acid are initially introduced into 410 g of dichloromethane. Then, at 50° C., 108.4 g of methanol and 9.6 g of p-toluenesulfonic acid are added and the mixture is stirred at reflux for 16 hours. When the reaction is complete, the reaction mixture is washed with water and sodium hydrogen carbonate solution, and the aqueous phases are extracted by shaking with chloroform and dried over magnesium sulfate. Filtration is then carried out, and the solvent is removed on a rotary evaporator and subjected to fractional distillation in vacuo. The product passes over at a temperature of 121-130° C. at 4 to 7 mbar. The resulting product has a saponification number of 360.0 mg

KOH/g (theory: 356.9 mg KOH/g) and a water content of <0.1% by weight. This gives 106.0 g of methyl N-methyl-2-pyrrolidone-4-carboxylate.

### Example 2

### Preparation of Isobutyl N-Methyl-2-Pyrrolidone-4-Carboxylate

507.8 g of N-methyl-2-pyrrolidone-4-carboxylic acid, 203.8 g of isobutanol and 10.0 g of p-toluenesulfonic acid are initially introduced into 500 g of chloroform and the mixture is stirred at reflux under a nitrogen atmosphere for 31 hours, during which the resulting water of reaction is continuously distilled off. When the reaction is complete, washing is carried out with sodium hydrogen carbonate solution, and the aqueous phase is extracted by shaking with chloroform and dried over magnesium sulfate. Filtration is then carried out followed by concentration on a rotary evaporator and fractional distillation in vacuo. The product passes over at a temperature of 143-155° C. at 5 to 7 mbar. The resulting product has a saponification number of 282.8 mg KOH/g (theory: 281.6 mg KOH/g) and a water content of <0.1% by weight. This gives 394.5 g of isobutyl N-methyl-2-pyrrolidone-4-carboxylate.

### Example 3

### Preparation of N-Butyl N-Methyl-2-Pyrrolidone-4-Carboxylate

363.5 g of dibutyl itaconate (M=242.3 g/mol) are initially introduced and heated to 70° C. with stirring under a nitrogen atmosphere. Then, 119.3 g of methylamine (40% strength by weight in water, M=31.1 g/mol) are added dropwise over the course of 2 hours, during which an exothermic reaction is observed. The reaction mixture is then brought to reflux temperature (103° C.) and stirred at reflux for 3 hours. Then, the water present and the butanol formed is distilled off at 110 to 160° C. for 2.5 hours. The crude product is subjected to fractional distillation in vacuo. The product passes over at a temperature of 162-170° C. at 8 mbar. The resulting product has a saponification number of 282.4 mg KOH/g (theory: 281.6 mg KOH/g) and a water content of <0.1% by weight. This gives 127.0 g of N-butyl N-methyl-2-pyrrolidone-4-carboxylate.

### Example 4

### Preparation of Methyl N-Butyl-2-Pyrrolidone-4-Carboxylate

200.0 g of dimethyl itaconate (M=158.2 g/mol) are initially introduced and heated to 50° C. with stirring under a nitrogen atmosphere. Then, 92.4 g of n-butylamine (M=73.1 g/mol) are added dropwise over the course of 20 minutes, during which an exothermic reaction is observed. The reaction mixture is then brought to reflux temperature (95° C.) and stirred at reflux for 6 hours. The methanol present is then distilled off at 100 to 130° C. for 1 hour. The crude product is subjected to fractional distillation in vacuo.

The product passes over at a temperature of 131° C. at 2 to 3 mbar. The resulting product has a saponification number of 282.6 mg KOH/g (theory: 281.6 mg KOH/g) and a water content of <0.1% by weight. This gives 175.2 g of methyl N-butyl-2-pyrrolidone-4-carboxylate.

### Example 5

### Preparation of Methyl N-Isobutyl-2-Pyrrolidone-4-Carboxylate

200.0 g of dimethyl itaconate (M=158.2 g/mol) are initially introduced and heated to 50° C. with stirring under a nitrogen atmosphere. Then, 92.4 g of isobutylamine (M=73.1 g/mol) are added dropwise over the course of 20 minutes, during which an exothermic reaction is observed. Then, the reaction mixture is brought to reflux temperature (98° C.) and stirred at reflux for 5 hours. The methanol formed is then distilled off at 100 to 120° C. for 1 hour. The crude product is subjected to fractional distillation in vacuo. The product passes over at a temperature of 142-144° C. at 5 mbar. The resulting product has a saponification number of 282.6 mg KOH/g (theory: 281.6 mg KOH/g) and a water content of <0.1% by weight. This gives 170.9 g of methyl N-isobutyl-2-pyrrolidone-4-carboxylate.

### Example 6

### Determination of the VOC Content

The VOC content of methyl N-methyl-2-pyrrolidone-4-carboxylate from example 1 and isobutyl N-methyl-2-pyrrolidone-4-carboxylate from example 2 is determined by a gas chromatographic measurement in accordance with DIN EN ISO 11890-2. GC conditions: separating column: 15 m Stabilwax, 0.53 mm ID, 1.0 μm film thickness; injector: split, split ratio 1:20; detector: FID; carrier gas: helium, 9 ml/min (40° C.), prepressure 22.4 kPa; detector gases: 350 ml/min synthetic air, 35 ml/min hydrogen, 21 ml/min helium (make-up gas); temperatures: injector: 250° C., detector: 280° C.; furnace: initial temperature: 40° C., holding time (isotherm): 3 min, heating rate: 25° C./min, end temperature: 260° C., holding time (isotherm): 5 min, injection volume: 2 μl; sample solution: ca. 1 g in 20 ml acetonitrile. Quantitative evaluation was carried out by means of calibration with an internal standard (isobutanol). Diethyl adipate (b.p. 251° C., Rₜ=8.8 min) was used as marker substances for the boiling point. All signals with a shorter retention time than diethyl adipate and also substances with a known boiling point <250° C. were evaluated. The VOC content is <0.2% by weight in both cases.

### Example 7

### Solubilities of Pesticides

The solubilities of various pesticides were determined in various compounds of the formula (1) at 25° C. All data are in % by weight (Table 1).

TABLE 1
Solubilities of pesticides in compounds of the formula (1)

| | | Pendi- | | | Tri- |
|---|---|---|---|---|---|
| Compound of the | Triflu- | meth- | Tebucon- | Triad- | floxy- |
| formula (1) | ralin | alin | azole | imefon | strobin |
| Methyl N-methyl- | 55.3 | 23.3 | 39.3 | 45.2 | 30.7 |
| 2-pyrrolidone-4- | | | | | |
| carboxylate from | | | | | |
| example 1 | | | | | |
| Isobutyl N-methyl- | 68.5 | 36.1 | 37.0 | 41.5 | 30.1 |
| 2-pyrrolidone- | | | | | |
| 4-carboxylate from | | | | | |
| example 2 | | | | | |
| N-butyl N-methyl- | 57.4 | 38.9 | 37.0 | 43.0 | 30.1 |
| 2-pyrrolidone- | | | | | |
| 4-carboxylate from | | | | | |
| example 3 | | | | | |
| Methyl N-butyl- | 58.6 | 38.9 | 38.2 | 43.0 | 31.1 |
| 2-pyrrolidone- | | | | | |
| 4-carboxylate from | | | | | |
| example 4 | | | | | |
| Methyl N-isobutyl- | 61.8 | 37.8 | 35.8 | 42.5 | 31.1 |
| 2-pyrrolidone- | | | | | |
| 4-carboxylate from | | | | | |
| example 5 | | | | | |

The result of the dissolution experiments shows that various, sparingly soluble pesticides with varying chemical structure are readily soluble in compounds of the formula (1).

1. A solvent comprising at least substituted 2-pyrrolidone-4-carboxylic acid ester of the formula (1) 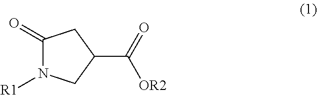
in which
R1 and R2 independently of one another are a linear, branched or cyclic C₁-C₆-alkyl.

2. The solvent as claimed in claim 1, wherein R1 and R2 in formula (1), independently of one another, are methyl, ethyl, n-propyl, isopropyl, n-butyl or isobutyl.

3. The solvent as claimed in claim 2, wherein, in formula (1)
R1 and R2 are methyl or
R1 is methyl and R2 is isobutyl or
R1 is n-butyl and R2 is methyl or
R1 is isobutyl and R2 is methyl.

4. The solvent as claimed in claim 1, wherein the VOC content of the compounds of the formula (1) in accordance with DIN EN ISO 11890-2 is less than 5% by weight.

5. (canceled)

6. (canceled)

7. (canceled)

8. (canceled)

9. (canceled)

10. (canceled)

11. (canceled)

12. A solvent for industrial applications comprising at least one N-substituted 2-pyrrolidone-4-carboxylic acid ester of the formula (1) 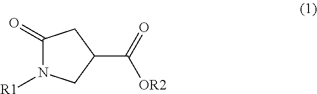
in which
R1 and R2 independently of one another are a linear, branched or cyclic C₁-C₆-alkyl.

13. A solvent for the manufacture of coatings and paints comprising at least one N-substituted 2-pyrrolidone-4-carboxylic acid ester of the formula (1) 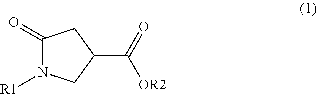
in which
R1 and R2 independently of one another are a linear, branched or cyclic C₁-C₆-alkyl.

14. A solvent for chemical synthesis comprising at least one N-substituted 2-pyrrolidone-4-carboxylic acid ester of the formula (1) 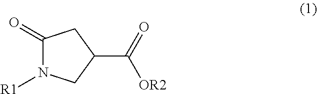
in which
R1 and R2 independently of one another are a linear, branched or cyclic C₁-C₆-alkyl.

15. A solvent for cleaning or degreasing, comprising at least one N-substituted 2-pyrrolidone-4-carboxylic acid ester of the formula (1) 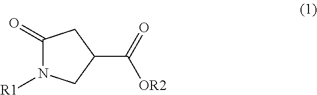
in which
R1 and R2 independently of one another are a linear, branched or cyclic C₁-C₆-alkyl.

16. A solvent for petrochemical processes comprising at least one N-substituted 2-pyrrolidone-4-carboxylic acid ester of the formula (1) 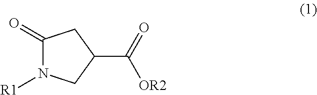
in which
R1 and R2 independently of one another are a linear, branched or cyclic C₁-C₆-alkyl.

17. A solvent for photolithography in the electronics and photovoltaic industry, comprising at least one N-substituted 2-pyrrolidone-4-carboxylic acid ester of the formula (1) 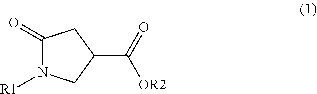
in which
R1 and R2 independently of one another are a linear, branched or cyclic C₁-C₆-alkyl.

18. A solvent for pesticide preparations comprising at least one N-substituted 2-pyrrolidone-4-carboxylic acid ester of the formula (1) 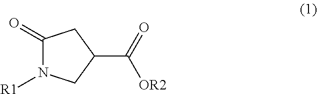
in which
R1 and R2 independently of one another are a linear, branched or cyclic C₁-C₆-alkyl.
